(19) United States
(12) Patent Application Publication
(10) Pub. No.: US 2003/0216441 A1
(43) Pub. Date: Nov. 20, 2003

(54) Farnesyltransferase inhibitors

(76) Inventors: Stephen L. Gwaltney II, Lindenhurst, IL (US); Lissa T.J. Nelson, Highland Park, IL (US); Stephen J. O'Connor, Guilford, CT (US); Hing L. Sham, Vernon Hills, IL (US); Gerard M. Sullivan, Round Lake Beach, IL (US); Weibo Wang, Moraga, CA (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/144,225

(22) Filed: May 10, 2002

(51) Int. Cl. A61K 31/5377; C07D 413/02; A61K 31/4439; C 07D 4 1/02

(52) U.S. Cl. 514/341; 514/235.2; 544/124; 546/272.7

(57) ABSTRACT

Compounds having the formula

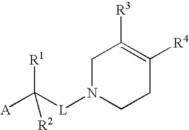

are farnesyltransferase inhibitors. Also disclosed are methods of making the compounds, pharmaceutical compositions containing the compounds, and methods of treatment using the compounds.

### TECHNICAL FIELD

The present invention provides substituted tetrahydropyridines which inhibit farnesyltransferase, methods for making the compounds, pharmaceutical compositions containing the compounds, and methods of treatment using the compounds.

### BACKGROUND OF THE INVENTION

Ras oncogenes are the most frequently identified activated oncogenes in human tumors, and transformed protein Ras is involved in the proliferation of cancer cells. The Ras must be farnesylated by farnesyl pyrophosphate before this proliferation can occur, and farnesylation of Ras by farnesyl pyrophosphate is effected by protein farnesyltransferase. Inhibition of protein farnesyltransferase, and thereby farnesylation of the Ras protein, blocks the ability of transformed cells to proliferate.

Activation of Ras and related proteins which are farnesylated also partially mediates smooth muscle cell proliferation. Inhibition of protein isoprenyl transferases, and thereby farnesylation of the Ras protein, also aids in the prevention of intimal hyperplasia associated with restenosis and atherosclerosis, a condition which compromises the success of angioplasty and surgical bypass for obstructive vascular lesions.

Because of its pivitol role in tumor formation and metastasis, there has been continued interest in finding compounds that inhibit farnesyltransferase.

### SUMMARY OF THE INVENTION

In its principle embodiment, the present invention provides a compound of formula (I)

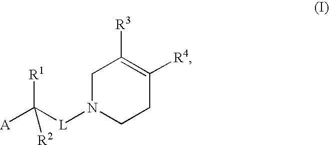

or a therapeutically acceptable salt thereof, wherein

A is selected from the group consisting of aryl and heteroaryl;

L is absent or selected from the group consisting of alkylene, N(R⁵), (CH₂)ₙC(O), (CH₂)ₙC(S), N(R⁵)(CH₂)ₙC(O), CH(NR⁵R⁶)C(O), and (CH₂)ₙSO₂; wherein n is 0-4; and

wherein each group is drawn with its left end attached to the carbon bearing R¹ and R² and its right end attached to the nitrogen;

R¹ and R² are independently selected from the group consisting of hydrogen, alkenyl, alkyl, alkynyl, aryl, arylalkenyl, arylalkyl, arylalkynyl, halo, and hydroxy; wherein the aryl and the aryl part of the arylalkenyl and the arylalkynyl can be optionally substituted with one, two, three, four, or five substituents independently selected from the group consisting of alkoxy, alkoxyalkyl, alkoxycarbonyl, alkyl, alkylcarbonyl, cyano, halo, haloalkoxy, haloalkyl, hydroxy, and nitro;

R³ and R⁴ are independently selected from the group consisting of hydrogen, alkoxy, alkoxycarbonyl, alkyl, aminocarbonyl, aryl, carboxy, cyano, halo, heteroaryl, heterocycle, and (heterocycle)carbonyl; and

R⁵ and R⁶ are independently selected from the group consisting of hydrogen, alkyl, aryl, and arylalkyl.

In a preferred embodiment, the present invention provides a compound of formula (I) wherein A is heteroaryl, L is absent, R¹ and R² are hydrogen, and R³ and R⁴ are as previously defined.

In another preferred embodiment, the present invention provides a compound of formula (I) wherein A is heteroaryl, L is absent, one of R¹ and R² is other than hydrogen, and R³ and R⁴ are as previously defined.

In another preferred embodiment, the present invention provides a compound of formula (I) wherein A is heteroaryl; L is (CH₂)ₙC(O); R¹ and R² are independently selected from the group consisting of hydrogen, alkenyl, alkyl, alkynyl, aryl, arylalkenyl, arylalkyl, arylalkynyl, halo, and hydroxy; wherein the aryl and the aryl part of the arylalkenyl and the arylalkynyl can be optionally substituted with one, two, three, four, or five substituents independently selected from the group consisting of alkoxy, alkoxyalkyl, alkoxycarbonyl, alkyl, alkylcarbonyl, cyano, halo, haloalkoxy, haloalkyl, hydroxy, and nitro; and R³, R⁴, and n are as previously defined.

In another preferred embodiment, the present invention provides a compound of formula (I) wherein A is heteroaryl; L is selected from the group consisting of N(R⁵)(CH₂)ₙC(O) and CH(NR⁵R⁵)C(O); R¹ and R² are independently selected from the group consisting of hydrogen, alkenyl, alkyl, alkynyl, aryl, arylalkenyl, arylalkyl, arylalkynyl, halo, and hydroxy; wherein the aryl and the aryl part of the arylalkenyl and the arylalkynyl can be optionally substituted with one, two, three, four, or five substituents independently selected from the group consisting of alkoxy, alkoxyalkyl, alkoxycarbonyl, alkyl, alkylcarbonyl, cyano, halo, haloalkoxy, haloalkyl, hydroxy, and nitro; and R³, R⁴, R⁵, R⁶, and n are as previously defined.

In another preferred embodiment, the present invention provides a compound of formula (I) wherein A is heteroaryl; L is selected from the group consisting of alkylene and N(R⁵); R¹ and R² are independently selected from the group consisting of hydrogen, alkenyl, alkyl, alkynyl, aryl, arylalkenyl, arylalkyl, arylalkynyl, halo, and hydroxy; wherein the aryl and the aryl part of the arylalkenyl and the arylalkynyl can be optionally substituted with one, two, three, four, or five substituents independently selected from the group consisting of alkoxy, alkoxyalkyl, alkoxycarbonyl, alkyl, alkylcarbonyl, cyano, halo, haloalkoxy, haloalkyl, hydroxy, and nitro; and R³, R⁴, R⁵, and n are as previously defined.

In another embodiment, the present invention provides a pharmaceutical composition comprising a compound of formula (I) or a therapeutically acceptable salt thereof, in combination with a therapeutically acceptable carrier.

In another embodiment, the present invention provides a method for inhibiting farnesyltransferase in a patient in recognized need of such treatment comprising administering to the patient a therapeutically acceptable amount of a compound of formula (I), or a therapeutically acceptable salt thereof.

In another embodiment, the present invention provides a method for treating cancer in a patient in recognized need of such treatment comprising administering to the patient a therapeutically acceptable amount of a compound of formula (I), or a therapeutically acceptable salt thereof.

### DETAILED DESCRIPTION OF THE INVENTION

As used herein, the singular forms “a”, “an”, and “the” include plural reference unless the context clearly dictates otherwise.

As used in the present specification the following terms have the meanings indicated:

The term “alkenyl,” as used herein, refers to a monovalent straight or branched chain group of one to six carbon atoms containing at least one carbon-carbon double bond.

The term “alkoxy,” as used herein, refers to an alkyl group attached to the parent molecular moiety through an oxygen atom.

The term “alkoxyalkyl,” as used herein, refers to an alkoxy group attached to the parent molecular moiety through an alkyl group.

The term “alkoxycarbonyl,” as used herein, refers to an alkoxy group attached to the parent molecular moiety through a carbonyl group.

The term “alkylene,” as used herein, refers to a divalent group derived from a straight or branched chain saturated hydrocarbon of one to six atoms.

The term “alkyl,” as used herein, refers to a group derived from a straight or branched chain saturated hydrocarbon of one to six atoms.

The term “alkylcarbonyl,” as used herein, refers to an alkyl group attached to the parent molecular moiety through a carbonyl group.

The term “alkynyl,” as used herein, refers to a group derived from a straight or branched chain hydrocarbon of two to six atoms containing at least one triple bond.

The term “amino,” as used herein, refers to —NR^{a}R^{b}, wherein R^{a} and R^{b} are independently selected from the group consisting of hydrogen, alkyl, alkylcarbonyl, cycloalkyl, (cycloalkyl)alkyl, and unsubstituted phenyl.

The term “aminoalkyl,” as used herein, refers to an amino group attached to the parent molecular moiety through an alkyl group.

The term “aminocarbonyl,” as used herein, refers to an amino group attached to the parent molecular moiety through a carbonyl group.

The term “aryl,” as used herein, refers to a phenyl group, or a bicyclic or tricyclic fused ring system wherein one or more of the fused rings is a phenyl group. Bicyclic fused ring systems are exemplified by a phenyl group fused to a monocyclic cycloalkyl group, as defined herein, or another phenyl group. Tricyclic fused ring systems are exemplified by a bicyclic fused ring system fused to a monocyclic cycloalkyl group, as defined herein, or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkenyl, alkoxy, alkoxyalkyl, alkoxycarbonyl, alkyl, alkylcarbonyl, amino, aminoalkyl, a second aryl group, arylalkyl, carboxy, cyano, halo, haloalkoxy, haloalkyl, hydroxy, hydroxyalkyl, nitro, and oxo; wherein the second aryl group and the aryl part of the arylalkyl can be further optionally substituted with one, two, three, four, or five substituents independently selected from the group consisting of alkenyl, alkoxy, alkoxyalkyl, carboxy, cyano, halo, haloalkoxy, haloalkyl, hydroxy, hydroxyalkyl, and nitro.

The term “arylalkenyl,” as used herein, refers to an aryl group attached to the parent molecular moiety through an alkenyl group.

The term “arylalkyl,” as used herein, refers to an aryl group attached to the parent molecular moiety through an alkyl group.

The term “arylalkynyl,” as used herein, refers to an aryl group attached to the parent molecular moiety through an alkynyl group.

The term “carbonyl,” as used herein, refers to —C(O)—.

The term “carboxy,” as used herein, refers to —CO₂H.

The term “cyano,” as used herein, refers to —CN.

The term “cycloalkyl,” as used herein, refers to a saturated monocyclic, bicyclic, or tricyclic hydrocarbon ring system having three to twelve carbon atoms. Examples of cycloalkyl groups include cyclopropyl, cyclopentyl, bicyclo[3.1.1]heptyl, adamantyl, and the like.

The term “(cycloalkyl)alkyl,” as used herein, refers to a cycloalkyl group attached to the parent molecular moiety through an alkyl group.

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heteroaryl,” as used herein, refers to an aromatic five- or six-membered ring where at least one atom is selected from the group consisting of N, O, and S, and the remaining atoms are carbon. The five-membered rings have two double bonds, and the six-membered rings have three double bonds. The heteroaryl groups are connected to the parent molecular group through a substitutable carbon or nitrogen atom in the ring. The term “heteroaryl” also includes bicyclic systems where a heteroaryl ring is fused to a phenyl group, a monocyclic cycloalkyl group, as defined herein, a heterocycle group, as defined herein, or an additional heteroaryl group; and tricyclic systems where a bicyclic system is fused to a phenyl group, a monocyclic cycloalkyl group, as defined herein, a heterocycle group, as defined herein, or an additional heteroaryl group. Heteroaryls are exemplified by benzothienyl, benzoxadiazolyl, cinnolinyl, dibenzofuranyl, furanyl, imidazolyl, indazolyl, indolyl, isoxazolyl, isoquinolinyl, isothiazolyl, naphthyridinyl, oxadiazolyl, oxadiazolyl, oxazolyl, thiazolyl, thienopyridinyl, thienyl, triazolyl, thiadiazolyl, pyridinyl, pyridazinyl, pyrimidinyl, pyrazinyl, pyrazolyl, pyrrolyl, quinolinyl, triazinyl, and the like. The heteroaryl groups of the present invention can be optionally substituted with one, two, three, four, or five substituents independently selected from the group consisting of alkenyl, alkoxy, alkoxyalkyl, alkoxycarbonyl, alkyl, alkylcarbonyl, amino, aminoalkyl, aryl, arylalkyl, cyano, halo, haloalkoxy, haloalkyl, hydroxy, hydroxyalkyl, and nitro; wherein the aryl group and the aryl part of the arylalkyl can be further optionally substituted with one, two, three, four, or five substituents independently selected from the group consisting of alkenyl, alkoxy, alkoxyalkyl, unsubstituted aryl, carboxy, cyano, halo, haloalkoxy, haloalkyl, hydroxy, hydroxyalkyl, and nitro.

The term “heterocycle,” as used herein, refers to cyclic, non-aromatic, five-, six-, or seven-membered rings containing at least one atom selected from the group consisting of oxygen, nitrogen, and sulfur. The five-membered rings have zero or one double bonds and the six- and seven-membered rings have zero, one, or two double bonds. The heterocycle groups of the invention are connected to the parent molecular group through a substitutable carbon or nitrogen atom in the ring. The term “heterocycle” also includes bicyclic systems where a heterocycle ring is fused to a phenyl group, a monocyclic cycloalkyl group, as defined herein, or an additional monocyclic heterocycle group; and tricyclic systems where a bicyclic system is fused to a phenyl group, a monocyclic cycloalkyl group, as defined herein, or an additional monocyclic heterocycle group. Heterocycle groups of the invention are exemplified by benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups of the present invention can be optionally substituted with one, two, three, four, or five substituents independently selected from the group consisting of alkenyl, alkoxy, alkoxyalkyl, alkoxycarbonyl, alkyl, alkylcarbonyl, amino, aminoalkyl, cyano, halo, haloalkoxy, haloalkyl, hydroxy, hydroxyalkyl, nitro, and oxo.

The term “(heterocycle)carbonyl,” as used herein, refers to a heterocycle group attached to the parent molecular moiety through a carbonyl group.

The term “hydroxy,” as used herein, refers to —OH.

The term “hydroxyalkyl,” as used herein, refers to a hydroxy group attached to the parent molecular moiety through an alkyl group.

The term “nitro,” as used herein, refers to —NO₂.

The term “oxo,” as used herein, refers to ═O.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate,trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, or mixtures thereof, which possess the ability to inhibit farnesyltransferase. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

In accordance with methods of treatment and pharmaceutical compositions of the invention, the compounds can be administered alone or in combination with other farnesyltransferase inhibitors. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The inhibitory effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

Farnesyltransferase Inhibition

Farnesyltransferase (FTase) or geranylgeranyltransferase I (GGTase I) fractions were isolated from bovine brains and purified by a series of methods which separate FTase from GGTase I and GGTase I from GGTase II. The methods involved a partial purification of all three enzymes by precipitation from a beef brain homogenate with 30% to 50% saturated (NH₄)₂SO₄ followed by chromatography on DEAE Sepharose. A Hydrophobic Interaction Chromatography (HIC) media, Fractogel-Phenyl (EM Industries) was used to separate FTase from GGTase; and chromatography of each enzyme on MonoQ (Pharmacia) resulted in further purification of the enzymes. The catalytic purity of each enzyme was assayed separately with substrate acceptor proteins specific for that enzyme. After quickly freezing in liquid nitrogen, the various prenyl transferases were stored at −80° C.

Bovine FTase was assayed at 37° C. for 30 minutes in a volume of 100 μL containing 44 mM HEPES, pH 7.4, 26 mM MgCl₂, 4.4 mM DTT, 18 mM KCl, 0.009% Triton X-100, 256 nM [³H]-farnesyl pyrophosphate, triammonium salt ([³H]-FPP, 759 GBq/mmol, New England Nuclear), 100 nM biotin-K-ras peptide (American Peptide Company), and FTase (12.5 μg/mL total protein). Reactions are initiated by the addition of FTase and stopped by the addition of 75 μL of a 1.43 mg/mL suspension of streptavidin SPA (Scintillation Proximity Assay) beads (Amersham) in 0.2M sodium phosphate, pH 4, containing 1.5M MgCl₂, 0.5% BSA and 0.05% sodium azide. The quenched reactions stood for 1 hour before analysis in a Packard TopCount scintillation counter. Purified compounds were dissolved in 100% ethanol and diluted 10-fold into the assay. The compounds of the present invention inhibited farnesyltransferase from about 50% to about 95% at concentrations of 10⁻⁹M with one compound inhibiting famesyltransferase at about 15% at a concentration of 10⁻⁸M. Preferred compounds inhibited famesyltransferase from about 85% to about 95% at concentrations of 10⁻⁹M.

As shown by these results, compounds of the present invention, including but not limited to those specified in the examples, are useful for the treatment of diseases caused or exascerbated by farnesyltransferase. As farnesyltransferase inhibitors, these compounds are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract (kidney, bladder, and urothelium); female genital tract (cervix, uterus, and ovaries); male genital tract (prostate, seminal vesicles, and testes); endocrine glands (thyroid, adrenal, and pituitary); skin (hemangiomas, melanomas, and sarcomas); tumors of the brain, nerves, and eyes; meninges (astrocytomas, gliomas, glioblastomas, retinoblastomas, neuromas, neuroblastomas, and meningiomas); solid tumors arising from hematopoietic malignancies (leukemias and chloromas); plasmacytomas; plaques; tumors of mycosis fungoides; cutaneous T-cell lymphoma/leukemia; lymphomas including Hodgkin's and non-Hodgkin's lymphomas; prophylaxis of autoimmune diseases (rheumatoid, immune and degenerative arthritis); ocular diseases (diabetic retinopathy, retinopathy of prematurity, corneal graft rejection, retrolental fibroplasia, neovascular glaucoma, rubeosis, retinal neovascularization due to macular degeneration, and hypoxia); skin diseases (psoriasis, hemagiomas and capillary proliferation within atherosclerotic plaques).

Synthetic Methods

Abbreviations which have been used in the descriptions of the scheme and the examples that follow are: PPh₃ for triphenylphosphine; DAST for (diethylamino)sulfur trifluoride; DMSO for dimethylsulfoxide; DMF for N,N-dimethylformamide; EDC for 1-(3-dimethylaminopropyl)-3-ethylcarbodiimide hydrochloride; DMAP for 4-dimethylaminopyridine; TFA for trifluoroacetic acid; HOBT for hydroxybenzotriazole; HATU for O-(7-azabenzotriazol-1-yl)-N,N,N′,N′-tetramethyluronium hexafluorophosphate; and THF for tetrahydrofuran.

The compounds and processes of the present invention will be better understood in connection with the following synthetic schemes which illustrate representative methods by which the compounds of the invention may be prepared. It will be readily apparent to one of ordinary skill in the art that the compounds of the invention can be prepared by a variety of synthetic routes. Other routes may be described in the examples. Starting materials can be obtained from commercial sources or prepared by well-established literature methods known to those of ordinary skill in the art. The groups A, L, R¹, R², R³, R⁴, R⁵, R⁶ and n are as defined above unless otherwise noted below.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

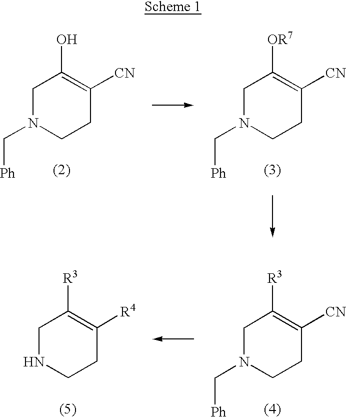

Scheme 1 shows the synthesis of compounds of formula (5). Compounds of formula (2) can be converted to compounds of formula (3) (where R⁷ is SO₂CF₃) by treatment with triflic anhydride in the presence of a base such as pyridine or diisopropylethylamine.

Compounds of formula (3) can be coupled with an appropriately substituted organometallic reagent (R³-M, where R³ is aryl, heteroaryl, or heterocycle and M is a substituted metal such as a boronic acid or a trialkylstannane) in the presence of a catalyst such as Pd(PPh₃)₄ or PdCl₂(PPh₃)₂ and optionally a base such as K₂PO₄ or K₂CO₃ to provide compounds of formula (4) where R³ is aryl, heteroaryl, or heterocycle. Alternatively, compounds of formula (3) can be treated with tributylamine, formic acid, and a palladium catalyst such as Pd(PPh₃)₄ to provide compounds of formula (4) where R³ is hydrogen.

Deprotection of compounds of formula (4) under conditions known to those of ordinary skill in the art (such as 1-chloroethylformate) provides compounds of formula (5) (where R³ is hydrogen, aryl, heteroaryl, or heterocycle and R⁴ is cyano).

Alternatively, compounds of formula (2) can be treated with an alkylating agent (such as diazomethane) to provide compounds of formula (3) where R⁷ is alkyl. These compounds can be deprotected using conditions known to those of ordinary skill in the art to provide compounds of formula (5) where R³ is alkoxy and R⁴ is cyano.

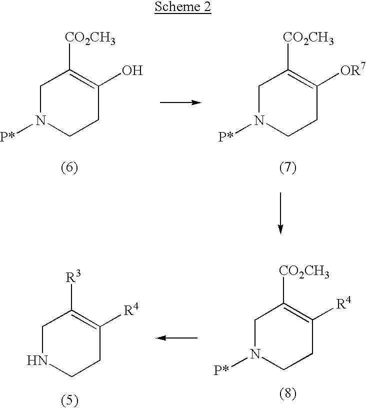

Compounds of formula (5) can also be prepared by the methods shown in Scheme 2. Compounds of formula (6) (where P* is a nitrogen protecting group such as tert-butoxycarbonyl; prepared by protecting the free amine by methods known to those of ordinary skill in the art) can be converted to compounds of formula (7) (where R⁷ is SO₂CF₃ or alkyl) using the methods described in Scheme 1.

Compounds of formula (7) where R⁷ is SO₂CF₃ can be converted to compounds of formula (8) (where R⁴ is hydrogen, aryl, heteroaryl, or heterocycle) using the methods described in Scheme 1.

Deprotection of compounds of formula (7) or (8) using conditions known to those of ordinary skill in the art (such as treatment with HCl when P* is tert-butoxycarbonyl) provides compounds of formula (5) (where R³ is alkoxycarbonyl and R⁴is alkoxy, hydrogen, aryl, heteroaryl, or heterocycle).

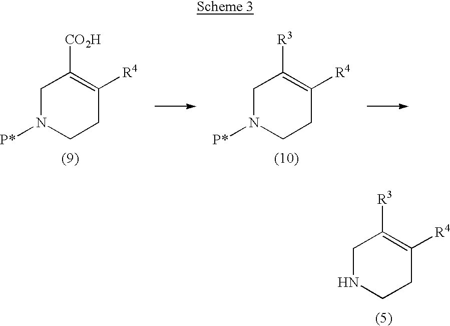

As shown in Scheme 3, compounds of formula (9) (where R⁴ is alkoxy, hydrogen, aryl, heteroaryl, or heterocycle; which can be prepared by hydrolysis of compounds of formula (7) or (8)) can be reacted with an appropriately substituted amine in the presence of a base such as diisopropylethylamine and a coupling reagent such as EDC, HATU, HOBT, and mixtures thereof to provide compounds of formula (9) (where R³ is aminocarbonyl or (heterocycle)carbonyl). These compounds can be subjected to deprotection conditions known to those of ordinary skill in the art to provide compounds of formula (5) (where R³ is aminocarbonyl or (heterocycle)carbonyl and R⁴ is alkoxy, hydrogen, aryl, heteroaryl, or heterocycle).

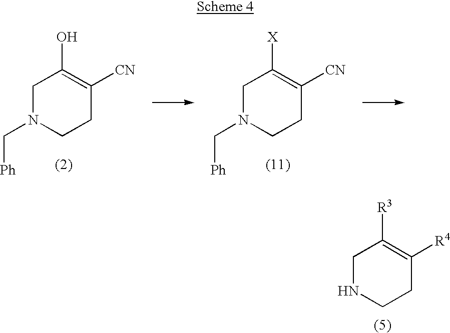

As shown in Scheme 4, compounds of formula (2) can be converted to compounds of compounds of formula (11) (where X is Br, Cl, F, or I) by treatment with the appropriate halogenating agent (POBr₃, POCl₃, DAST, or PPh₃/I₂, respectively).

Deprotection of compounds of formula (11) under conditions known to those of ordinary skill in the art (such as 1-chloroethylformate) provides compounds of formula (5) (where R³ is halo and R⁴ is cyano).

This sequence can also be carried out on compounds of formula (6) (shown in Scheme 2) to provide compounds of formula (5) where R³ is alkoxycarbonyl and R⁴is halo.

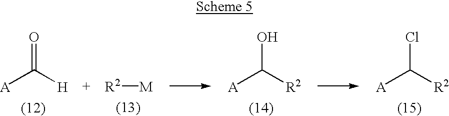

As shown in Scheme 5, compounds of formula (12) can be treated with compounds of formula (13) (where R² is alkenyl, alkynyl, arylalkenyl, or arylalkynyl and M is a metal such as MgBr or Li) to provide compounds of formula (14) which can be converted to compounds of formula (15) by treatment with a chlorinating agent such as thionyl chloride.

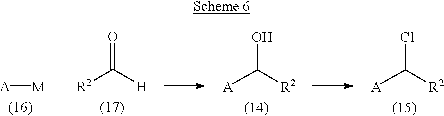

Compounds of formula (17) (where R² is alkyl or arylalkyl) can be treated with compounds of formula (16) (where M is a metal such as MgBr or Li) to provide compounds of formula (14) which can be converted to compounds of formula (15) by treatment with a chlorinating agent such as thionyl chloride.

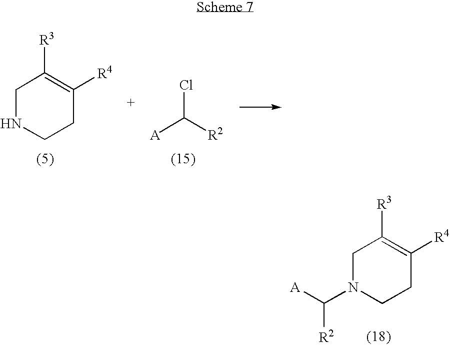

Scheme 7 shows the synthesis of compounds of formula (18) (compounds of formula (I) where L is absent and R¹ is hydrogen). Compounds of formula (5) can be treated with compounds of formula (15) in the presence of a base such as diisopropylethylamine to provide compounds of formula (18).

Compounds of formula (18) where R³ is alkoxycarbonyl can be hydrolyzed under conditions known to those of ordinary skill in the art (for example, LiOH) to provide compounds of formula (18) where R³ is carboxy.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names which appeared to be consistent with ACD nomenclature.

### EXAMPLE 1

4-cyano-N-(4-cyanobenzyl)-N-[(1-methyl-1H-imidazol-5-yl)methyl]-5-(1-naphthyl)-3,6-dihydropyridine-1(2H)-carboxamide

### EXAMPLE 1A

ethyl[benzyl(3-cyanopropyl)amino]acetate

A mixture of N-benzylglycine ethyl ester (3 mL, 16 mmol), 4-bromobutyronitrile (1.6 mL, 16 mmol), and powdered K₂CO₃ (2.65 g, 19.2 mmol) at 100° C. was stirred for about 16 hours, cooled to room temperature, and partitioned between water and dichloromethane. The aqueous phase was extracted with dichloromethane and the combined extracts were dried (MgSO₄), filtered, and concentrated. The concentrate was purified by flash column chromatography on silica gel with 10 to 20% ethyl acetate/hexanes to provide the desired product (3.04 g, 73%). MS (DCI(NH₃) m/e 261 (M+H)⁺; ¹H NMR(300 MHz, CDCl₃) δ7.31 (m, 5H), 4.16 (q, J=7.0 Hz, 2H), 3.77 (s, 2H), 3.30 (s, 2H), 2.78 (t, J=6.6 Hz, 2H), 2.45 (t, J=7.4 Hz, 2H), 1.80 (p, J=7.0 Hz, 2H), 1.27 (t, J=7.0 Hz, 3H).

### EXAMPLE 1B

1-benzyl-5-hydroxy-1,2,3,6-tetrahydropyridine-4-carbonitrile

A suspension of potassium tert-butoxide (591 mg, 5.28 mmol) in toluene (15 mL) at 70° C. was treated with a solution of Example 1A (549 mg, 2.11 mmol) in toluene (5 mL), stirred for 25 minutes, cooled to room temperature, poured into 5.5M NH₄Cl, and extracted twice with 20% isopropanol/chloroform. The combined extracts were dried (MgSO₄), filtered, and concentrated. Residual isopropanol was removed by azeotropic distillation with toluene. The residue was dried under vacuum overnight to provide the desired product (378 mg, 84%). MS (DCI/NH₃) m/e 215 (M+H)⁺.

### EXAMPLE 1C

1-benzyl-5-(1-naphthyl)-1,2,3,6-tetrahydropyridine-4-carbonitrile

A solution of Example 1B (377 mg, 1.76 mmol) in dichloromethane (10 mL) and pyridine (0.43 mL, 5.3 mmol) at 0° C. was treated with triflic anhydride (0.44 mL, 2.64 mmol), stirred for about 2 hours, poured into saturated NaHCO₃, and extracted twice with dichloromethane. The combined extracts were dried (MgSO₄), filtered, and concentrated. The concentrate was purified on a one inch plug of silica gel with 40% ethyl acetate/hexanes to provide the triflate, which was used without further purification.

A solution of the triflate (359 mg, 1.04 mmol) in dry dioxane (6 mL) was treated with powdered K₂PO₄ tribasic (442 mg, 2.08 mmol), 1-naphthylboronic acid (268 mg, 1.56 mmol), and Pd(PPh₃)₄ (60 mg, 0.052 mmol), purged three times with nitrogen, and heated to 85° C. for 18 hours. The reaction was cooled to room temperature, poured into 2M Na₂CO₃, and extracted with ethyl acetate. The combined extracts were washed with brine, dried (MgSO₄), filtered, and concentrated. The concentrate was purified by flash column chromatography on silica gel with 10% to 20% ethyl acetate/hexanes to provide the desired product (240 mg, 42% for the 2 step process). MS (ESI(+)) m/e 325 (M+H)⁺; ¹H NMR(300 MHz, CDCl₃) δ7.77 (m, 2H), 7.69 (m, 1H), 7.40 (m, 3H), 7.23 (m, 6H), 3.62(s, 2H), 3.47 (m, 1H), 3.19 (m, 1H), 2.82 (m, 1H), 2.68 (m, 2H), 2.53 (m, 1H).

### EXAMPLE 1D

5-(1-naphthyl)-1,2,3,6-tetrahydropyridine-4-carbonitrile hydrochloride

A solution of Example 1C (405 mg, 1.25 mmol) in dichloroethane (10 mL) at reflux was treated with 1-chloroethylchloroformate (0.3 mL, 2.5 mmol), stirred for 2 hours, cooled to room temperature, and concentrated. The concentrate was treated with methanol (10 mL), stirred at 50° C. for 2.5 hours, cooled to room temperature, and concentrated. The concentrate was suspended in dichloromethane, treated with diethyl ether, filtered, and dried under high vacuum to provide the desired product (302 mg, 89%). MS (DCI/NH₃) m/e 235 (M+H)⁺; ¹H NMR (500 MHz, DMSO-d₆) δ9.90 (s, 2H), 8.05 (m, 3H), 7.62 (m, 3H), 7.48 (dd, J=1.1, 7.3 Hz, 1H), 4.05 (m, 2H), 3.56 (m, 1H), 3.38 (m, 1H), 2.89 (m, 1H), 2.75 (m, 1H).

### EXAMPLE 1E

4-({[(1-methyl-1H-imidazol-5-yl)methyl]amino}methyl)benzonitrile

A mixture of 4-cyanobenzyl amine (prepared according to the procedure described in WO 00/01691, 310 mg, 2.35 mmol) and 1-methyl-1H-imidazole-5-carbaldehyde (prepared according to the procedure described in J. Org. Chem. 1993, 58, 1159-1166, 258 mg, 2.35 mmol) in dichloromethane (15 mL) was treated with acetic acid (2 drops), stirred for 40 minutes, treated with sodium triacetoxyborohydride (1 g, 4.7 mmol), and stirred overnight. The mixture was treated with methanol (15 mL), stirred for two hours, and concentrated. The concentrate was partitioned between saturated NaHCO₃ and dichloromethane and the aqueous phase was extracted twice with dichloromethane. The combined extracts were dried (MgSO₄), filtered, and concentrated. The concentrate was purified by flash column chromatography on silica gel with 10% methanol/dichloromethane to provide the desired product (270 mg, 51%). MS (DCI(NH₃) m/e 227 (M+H)⁺; ¹H NMR (300 MHz, CDCl₃) δ7.63 (d, J=8.1 Hz, 2H), 7.45 (m, 3H), 6.93 (s, 1H), 3.86 (s, 2H), 3.76 (s, 2H), 3.67 (s, 3H).

### EXAMPLE 1F

4-cyano-N-(4-cyanobenzyl)-N-[(1-methyl-1H-imidazol-5-yl)methyl]-5-(1-naphthyl)-3,6-dihydropyridine-1(2H)-carboxamide

A solution of Example 1E (99 mg, 0.44 mmol) in dichloroethane (4 mL) at 0° C. was treated with lutidine (0.15 mL) and triphosgene (52 mg, 0.18 mmol), stirred for 30 minutes, warmed to room temperature, stirred for 15 minutes, treated with the free base of Example 1D (generated by partitioning Example 1D between ethyl acetate and saturated sodium bicarbonate, then concentrating the organic layer) (103 mg, 0.44 mmol), and stirred overnight. The reaction was heated to 60° C. for 5 hours, cooled to room temperature, poured into 2M Na₂CO₃, and extracted twice with dichloromethane. The combined extracts were dried (MgSO₄), filtered, and concentrated. The concentrate was purified by flash column chromatography on silica gel with 5 to 20% ethanol/ethyl acetate containing 1% NH₄OH to provide the desired product (124 mg, 58%). MS (ESI(+)) m/e 487 (M+H)⁺; ¹H NMR (300 MHz, CH₃OD) δ8.85 (m, 1H), 7.97 (m, 2H), 7.81 (mn, 1H), 7.68 (m, 2H), 7.56 (m, 3H), 7.42 (m, 4H), 4.60 (m, 2H), 4.47 (m, 2H), 4.25 (m, 2H), 3.83 (s, 3H), 3.79 (m, 1H), 1H); Anal. Calcd. for C₃₀H₂₆N₆O.HCl.2.2H₂O: C, 64.04; H, 5.62; N, 14.94. Found: C, 63.94; H, 5.32; N, 15.08.

### EXAMPLE 2

1-({(4-cyanobenzyl)[(1-methyl-1H-imidazol-5-yl)methyl]amino}acetyl)-5-(1-naphthyl)-1,2,3,6-tetrahydropyridine-4-carbonitrile

### EXAMPLE 2A

1-{[(4-cyanobenzyl)amino]acetyl}-5-(1-naphthyl)-1,2,3,6-tetrahydropyridine-4-carbonitrile

A solution of Example 1D (503 mg, 1.86 mmol) in DMF (10 mL) at room temperature was treated with triethylamine (0.5 mL), N-Boc-glycine (360 mg, 2.05 mmol), EDC (536 mg, 2.80 mmol), and DMAP (20 mg), stirred overnight, diluted with ethyl acetate, washed sequentially with 1M NaHSO₄, water, saturated NaHCO₃, and brine, dried (MgSO₄), filtered, and concentrated. The concentrate was treated with 4M HCl in dioxane (3 mL), stirred for 3 hours and concentrated to provide the amino amide which was used immediately in the next reaction.

A solution of the amino amide (560 mg, 1.71 mmol) in dichloromethane (10 mL) at room temperature was treated with 4-cyanobenzaldehyde (245 mg, 1.88 mmol) and sodium triacetoxyborohydride (725 mg, 3.42 mmol), stirred overnight, diluted with dichloromethane, washed with saturated NaHCO₃, dried (MgSO₄), filtered, and concentrated. The concentrate was purified by flash column chromatography on silica gel with 1% methanol/dichloromethane to provide the desired product (334 mg, 48%). MS (ESI(+)) m/e 407 (M+H)⁺.

### EXAMPLE 2B

1-({(4-cyanobenzyl)[(1-methyl-1H-imidazol-5-yl)methyl]amino}acetyl)-5-(1-naphthyl)-1,2,3,6-tetrahydropyridine-4-carbonitrile

A solution of Example 2A (66 mg, 0.16 mmol) in dichloromethane (3 mL) at room temperature was treated with 1-methyl-1H-imidazole-5-carbaldehyde (prepared according to the procedure described in J. Org. Chem. 1993, 58, 1159-1166, 28 mg, 0.24 mmol) and sodium triacetoxyborohydride (86 mg, 0.4 mmol), stirred overnight, diluted with dichloromethane, washed with saturated NaHCO₃, dried (MgSO₄), and concentrated. The concentrate was purified by flash column chromatography on silica gel with 2% methanol/dichloromethane to provide the desired product (334 mg, 48%). MS (ESI(+)) m/e 501 (M+H)⁺.

### EXAMPLE 3

1-[(1-methyl-1H-imidazol-5-yl)methyl]-5-(1-naphthyl)-1,2,3,6-tetrahydropyridine-4-carbonitrile

The desired product was prepared as the bis-trifluoroacetate salt (82 mg, 38%) by substituting Example 1D (106 mg, 0.39 mmol) for Example 2A and using 43 mg (0.39 mmol) of 1-methyl-1H-imidazole-5-carbaldehyde (prepared according to the procedure described in J. Org. Chem. 1993, 58, 1159-1166) in Example 2B. MS (ESI(+)) m/e 329 (M+H)⁺; ¹H NMR (300 MHz, CH₃OD) δ8.90 (s, 1H), 7.94 (m, 2H), 7.80 (m, 1H), 7.54 (m, 4H), 7.39 (m, 1H), 4.01 (s, 3H), 3.90 (s, 2H), 3.65−3.43 (m, 2H), 3.06−2.86 (m, 2H), 2.76−2.58 (m, 2H); Anal. Calcd. for C₂₁H₂₀N₄.2TFA.2.66H₂O: C, 49.68; H, 4.56; N, 9.27. Found: C, 49.63; H, 4.16; N, 9.24.

### EXAMPLE 4

1-[(4-cyanophenyl)(1-methyl-1H-imidazol-5-yl)methyl]-5-(1-naphthyl)-1,2,3,6-tetrahydropyridine-4-carbonitrile

### EXAMPLE 4A

4-[hydroxy(1-methyl-1H-imidazol-5-yl)methyl]benzonitrile

A solution of 2-triethylsilylimidazole (prepared according to the procedure described in Tet. Lett. 1996, 37, 9353-9356; 3.5 mL, 16.8 mmol) in THF (50 mL) at −78° C. was treated slowly with 1.7M tert-butyllithium in pentane (9.9 mL, 16.8 mmol) stirred for thirty minutes, treated slowly with 4-cyanobenzaldehyde (2.09 g, 16 mmol) in THF (7 mL), stirred for 1 hour, quenched with methanol (4 mL), treated with 1N HCl (20 mL), and stirred overnight. The mixture was partitioned between ethyl acetate and saturated sodium bicarbonate. The organic layer was dried (MgSO₄), filtered, and concentrated. The concentrate was triturated with 20% ethyl acetate/hexanes to provide the desired product (2.57 g, 75%). MS (DCI/NH₃) m/e 214 (M+H)⁺; ¹H NMR (300 MHz, CDCl₃) δ7.67 (d, J=8.5 Hz, 2H), 7.54 (d, J=8.1 Hz, 2H), 7.40 (s, 1H), 6.66 (s, 1H), 5.95 (s, 1H), 3.53 (s, 3H).

### EXAMPLE 4B

4-[chloro(1-methyl-1H-imidazol-5-yl)methyl]benzonitrile hydrochloride

A solution of Example 4A (250 mg) in dichloromethane (10 mL) was treated with thionyl chloride (10 eq.), stirred for 2 hours, and concentrated to provide the desired product.

### EXAMPLE 4C

1-[(4-cyanophenyl)(1-methyl-1H-imidazol-5-yl)methyl]-5-(1-naphthyl)-1,2,3,6-tetrahydropyridine-4-carbonitrile

A suspension of Example 4B (99 mg, 0.37 mmol) and Example 1D (100 mg, 0.37 mmol) in acetonitrile (3 mL) was treated with diisopropylethylamine (0.32 mL), heated to 50° C., stirred overnight, and cooled to room temperature. The mixture was partitioned between saturated sodium bicarbonate and dichloromethane and the organic layer was dried (MgSO₄), filtered, and concentrated. The concentrate was purified by HPLC with 1 to 70% acetonitrile/water containing 0.1% TFA to provide the desired product as the bis-trifluoroacetate salt (76 mg, 31%). MS (ESI(+)) m/e 430 (M+H)⁺; ¹H NMR (300 MHz, CH₃OD) δ8.87 (m, 1H), 7.91 (m, 2H), 7.79 (m, 2H), 7.69 (m, 4H), 7.59−7.46 (m, 3H), 7.36−7.28 (m, 1H), 5.27 (m, 1H), 3.88 (s, 3H), 3.67−3.59 (m, 1H), 3.41 (m, 1H), 3.01 (m, 1H), 2.87−2.74 (m, 2H), 2.63 (m, 1H). Anal. Calcd. for C₂₈H₂₃N₅.1.76TFA: C, 60.07; H, 3.96; N, 11.11. Found: C, 59.95; H, 4.17; N, 11.43.

### EXAMPLE 5

1-{[1-(4-cyanobenzyl)-1H-imidazol-5-yl]methyl}-5-(1-naphthyl)-1,2,3,6-tetrahydropyridine-4-carbonitrile

The desired product was prepared by substituting Example 1D (69 mg, 0.26 mmol) and 4-[(5-formyl-1H-imidazol-1-yl)methyl]benzonitrile (prepared according to the procedure described in J. Med. Chem. 1999, 42, 3779-3784; 54 mg, 0.26 mmol) for Example 2A and 1-methyl-1H-imidazole-5-carbaldehyde, respectively, in Example 2B. Purification by HPLC with 1 to 70% acetonitrile/water containing 0.1% TFA provided the desired product as the bis-trifluoroacetate salt (63 mg, 37%). MS (ESI(+)) m/e 430 (M+H)⁺; ¹H NMR (300 MHz, CH₃OD) δ9.11 (d, J=1.7 Hz, 1H), 7.93 (m, 2H), 7.81 (m, 2H), 7.70 (m, 1H), 7.63 (d, J=1.4 Hz, 1H), 7.58−7.50 (m, 5H), 7.31 (dd, J=1.4, 7.1 Hz, 1H), 5.72 (s, 2H), 3.71 (s, 2H), 3.37 (m, 2H), 2.84 (m, 1H), 2.76 (m, 1H), 2.52 (m, 1H), 2.45 (m, 1H). Anal. Calcd. for C₂₈H₂₃N₅.2 TFA: C, 58.45; H, 3.83; N, 10.65. Found: C, 58.24; H, 4.02; N, 10.77.

### EXAMPLE 6

1-[1-(1-methyl-1H-imidazol-5-yl)-3-phenylprop-2-ynyl]-5-(1-naphthyl)-1,2,3,6-tetrahydropyridine-4-carbonitrile

### EXAMPLE 6A

1-(1-methyl-1H-imidazol-5-yl)-3-phenylprop-2-yn-1-ol

A suspension of 1-methyl-1H-imidazole-5-carbaldehyde (prepared according to the procedure described in J. Org. Chem. 1993, 58, 1159-1166, 170 mg, 1.55 mmol) in dioxane (10 mL) was treated with 1M phenylethynylmagnesium bromide in THF (7.73 mL, 7.73 mmol), heated to 80° C. for 1 hour, cooled to room temperature, treated with brine, and extracted with ethyl acetate. The organic phase was dried (MgSO₄), filtered, and concentrated. The concentrate was purified by flash column chromatography on silica gel with 2% methanol/ethyl acetate with 0.2% NH₄OH to provide the desired product (210 mg, 64%). MS (DCI/NH₃) m/e 213 (M+H)⁺; ¹H NMR (300 MHz, CDCl₃) δ7.52 (s, 1H), 7.47 (m, 2H), 7.34 (m, 3H), 7.15 (s, 1H), 5.74 (s, 1H), 3.83 (s, 3H).

### EXAMPLE 6B

1-[1-(1-methyl-1H-imidazol-5-yl)-3-phenylprop-2-ynyl]-5-(1-naphthyl)-1,2,3,6-tetrahydropyridine-4-carbonitrile

A solution of Example 6A (106 mg, 0.5 mmol) in dichloromethane (4 mL) at room temperature was treated with thionyl chloride (0.4 mL, 5 mmol), stirred for 2 hours, and concentrated. The concentrate was treated with a mixture of Example 1D (123 mg, 0.45 mmol), acetonitrile (4 mL), and diisopropylethylamine (0.44 mL), heated to 50° C. overnight, cooled to room temperature, and partitioned between saturated sodium bicarbonate and dichloromethane. The organic layer was dried (MgSO₄), filtered, and concentrated. The concentrate was purified by HPLC with 1 to 70% acetonitrile/water containing 0.1% TFA to provide the desired product (39 mg, 13%) as the bis-trifluoroacetate salt. MS (ESI(+)) m/e 429 (M+H)⁺; ¹H NMR (300 MHz, CH₃OD) δ8.97 (s, 1H), 7.94 (d, J=8.5 Hz, 2H), 7.70 (m, 1H), 7.53 (m, 6H), 7.40 (m, 4H), 5.49 (m, 1H), 4.11 (m, 3H), 3.92−3.48 (m, 3H), 3.14−3.01 (m, 2H), 2.78−2.68 (m, 2H); Anal. Calcd. for C₂₉H₂₄N₄.1.41TFA: C, 64.85; H, 4.35; N, 9.51. Found: C, 64.83; H, 4.64; N, 9.42.

### EXAMPLE 7

1-[(2R)-2-[(4-cyanobenzyl)amino]-3-(1-methyl-1H-imidazol-5-yl)propanoyl]-5-(1-naphthyl)-1,2,3,6-tetrahydropyridine-4-carbonitrile

### EXAMPLE 7A

tert-butyl (1R)-2-[4-cyano-5-(1-naphthyl)-3,6-dihydropyridin-1(2H)-yl]-1-[(1-methyl-1H-imidazol-5-yl)methyl1-2-oxoethylcarbamate

A solution of (2R)-2-[(tert-butoxycarbonyl)amino]-3-(1-methyl-1H-imidazol-5-yl)propanoic acid (219 mg, 0.815 mmol) and Example 1D (220 mg, 0.815 mmol) in DMF (5 mL) at 0° C. was treated with HOBT (121 mg, 0.9 mmol), diisopropylethylamine (0.2 mL), and EDC (172 mg, 0.9 mmol). The mixture was stirred overnight, treated with HOBT (121 mg) and EDC (172 mg), stirred for 24 hours, and partitioned between ethyl acetate and saturated sodium bicarbonate. The organic layer was dried (MgSO₄), filtered, and concentrated to provide the desired product. MS (ESI(+)) m/e 486 (M+H)⁺.

### EXAMPLE 7B

1-[(2R)-2-amino-3-(1-methyl-1H-imidazol-5-yl)propanoyl]-5-(1-naphthyl)-1,2,3,6-tetrahydropyridine-4-carbonitrile hydrochloride

Example 7A was treated with 4M HCl in dioxane (5 mL), stirred overnight, and concentrated to provide the desired product. MS (ESI(+)) m/e 386 (M+H)⁺ (free base).

### EXAMPLE 7C

1-[(2R)-2-[(4-cyanobenzyl)amino]-3-(1-methyl-1H-imidazol-5-yl)propanoyl]-5-(1-naphthyl)-1,2,3,6-tetrahydropyridine-4-carbonitrile

Example 7B was treated with a mixture of 4-cyanobenzaldehyde (214 mg, 1.63 mmol) in dichloromethane (10 mL) and methanol (1 mL), treated with sodium triacetoxyborohydride (518 mg, 2.45 mmol), and stirred for 3 days. The mixture was partitioned between saturated sodium bicarbonate and ethyl acetate and the organic layer was dried (MgSO₄), filtered, and concentrated. Half of the crude material was purified by HPLC with 1 to 70% acetonitrile/water containing 0.1% TFA to provide the desired product (126 mg) as the bis-trifluoroacetate salt. MS (ESI(+)) m/e 501 (M+H)⁺; Anal. Calcd. for C₃₁H₂₈N₆O.2TFA.2.22H₂O: C, 54.69; H, 4.52; N, 10.93. Found: C, 54.67; H, 4.21; N, 10.86.

### EXAMPLE 8

1-{[-(4-cyanobenzyl)-1H-imidazol-5-yl]acetyl}-5-(1-naphthyl)-1,2,3,6-tetrahydropyridine-4-carbonitrile

A solution of 1-(4-cyanophenylmethyl)-1H-imidazol-5-ylacetic acid (prepared according to the procedure described in J. Med. Chem. 1999, 42, 3356-3368; 1.12 mmol) and Example 1D (302 mg, 1.12 mmol) in DMF (5 mL) and diisopropylethylamine (0.4 mL) at room temperature was treated with HATU (511 mg, 1.34 mmol), stirred for 3 days, and partitioned between ethyl acetate and saturated sodium bicarbonate. The organic layer was dried (MgSO₄), filtered, and concentrated. The concentrate was purified by HPLC with 1 to 70% acetonitrile/water containing 0.1% TFA to provide the desired product (300 mg, 47%) as the trifluoroacetate salt. MS (ESI(+)) m/e 458 (M+H)⁺; ¹H NMR (300 MHz, CH₃OD) δ8.97 (m, 1H), 8.00−7.91 (m, 2H), 7.84−7.70 (m, 3H), 7.61−7.41 (m, 7H), 5.55−5.48 (m, 2H), 4.58−4.25 (m, 2H), 3.98−3.74 (m, 2H), 3.1−2.9 (m, 2H), 2.9−2.6 (m, 2H); Anal. Calcd. for C₂₉H₂₃N₅O.1TFA.2.38H₂O: C, 60.60; H, 4.72; N, 11.40. Found: C, 60.54; H, 4.35; N, 11.48.

### EXAMPLE 9

1-[2-(4-cyanophenyl)-2-hydroxy-2-(1-methyl-1H-imidazol-5-yl)ethyl]-5-(1-naphthyl)-1,2,3,6-tetrahydropyridine-4-carbonitrile

### EXAMPLE 9A

1-[2-(4-cyanophenyl)-2-oxoethyl]-5-(1-naphthyl)-1,2,3,6-tetrahydropyridine-4-carbonitrile

A solution of Example 1D (1.44 g, 5.33 mmol) and 4-cyanophenacyl bromide (1.2 g, 5.33 mmol) in DMF (10 mL) and diisopropylethylamine (2 mL) at room temperature was stirred overnight and partitioned between ethyl acetate and saturated sodium bicarbonate. The organic layer was dried (MgSO₄), filtered, and concentrated to provide the desired product. MS (ESI(+)) m/e 378 (M+H)⁺; ¹H NMR (400 MHz, CDCl₃) δ8.06 (m, 2H), 7.86 (m, 2H), 7.75 (m, 3H), 7.48 (m, 3H), 7.33 (dd, J=1.3, 6.8 Hz, 1H), 4.00 (d, J=4.7 Hz, 2H), 3.70 (m, 1H), 3.46 (m, 1H), 3.07 (m, 1H), 2.97 (m, 1H).

### EXAMPLE 9B

1-[2-(4-cyanophenyl)-2-hydroxy-2-(1-methyl-1H-imidazol-5-yl)ethyl[-5-(1-naphthyl)-1,2,3,6-tetrahydropyridine-4-carbonitrile

A suspension of CeCl₃ (120 mg, 0.49 mmol) in THF (1 mL) at room temperature was stirred overnight. A solution of 2-triethylsilylimidazole (0.48 mmol) in THF (2 mL) at −78° C. was treated slowly with 1.7M tert-butyllithium in pentane (0.28 mL, 0.48 mmol) and stirred for thirty minutes. The suspension of CeCl₃ was cooled to −78° C. and treated with the solution of the lithiated imidazole, warmed to −30° C., cooled to −78° C., treated slowly with a solution of Example 9A (164 mg, 0.44 mmol) in THF (1.5 mL), warmed to 0° C., quenched with 1N HCl, and partitioned between ethyl acetate and saturated sodium bicarbonate. The organic layer was dried (MgSO₄), filtered, and concentrated. The concentrate was purified by flash column chromatography on silica gel with 10% methanol/ethyl acetate with 0.5% NH₄OH, dissolved in acetonitrile (5 mL), treated with 1N HCl (10 mL), and lyophilized to provide the desired product (116 mg, 50%) as the dihydrochloride salt. MS (ESI(+)) m/e 460 (M+H)⁺; ¹H NMR (300 MHz, CH₃OD) δ8.95 (d, J=1.0 Hz, 1H), 7.98−7.90 (m, 4H), 7.79 (m, 4H), 7.61−7.52 (m, 3H), 7.38 (m, 1H), 4.35 (m, 1H), 4.21 (m, 1H), 4.10 (m, 1H), 3.90 (m, 1H), 3.75 (m, 1H), 3.59 (s, 3H), 3.45 (m, 1H), 2.9 (m, 2H); Anal. Calcd. for C₂₉H₂₅N₅O.2HCl.1.64H₂O: C, 61.98; H, 5.43; N, 12.46. Found: C, 61.98; H, 5.45; N, 12.46.

### EXAMPLE 10

1-({1-[4-cyano-3-(1-naphthyl)benzyl]-1H-imidazol-5-yl}acetyl)-5-methoxy-1,2,3,6-tetrahydropyridine-4-carbonitrile

### EXAMPLE 10A

methyl{1-[4-cyano-3-(1-naphthyl)benzyl]-1H-imidazol-5-yl}acetate

A mixture of methyl (1-trityl-1H-imidazol-5-yl)acetate (prepared according to the procedure described in J. Med. Chem. 1999, 42, 3356-3368; 669 mg, 1.75 mmol) and 4-(bromomethyl)-2-(1-naphthyl)benzonitrile (prepared according to the procedure described in WO 01/81316; 564 mg, 1.75 mmol) in ethyl acetate (10 mL) was heated to reflux for two days, cooled to room temperature, and treated with diethyl ether (50 mL). The mixture was filtered and the filter cake was dissolved in methanol (10 mL), heated to 60° C. for 3 days, cooled to room temperature, and concentrated. The concentrate was partitioned between ethyl acetate and saturated sodium bicarbonate and the organic layer was dried (MgSO₄), filtered, and concentrated. The concentrate was purified by flash column chromatography on silica gel with 1 to 2% methanol/ethyl acetate with 0.2% NH₄OH to provide the desired product (314 mg, 47%). MS (APCI) m/e 382 (M+H)⁺; ¹H NMR (300 MHz, CDCl₃) δ7.95 (m, 2H), 7.81 (d, J=7.8 Hz, 1H), 7.58−7.48 (m, 3H), 7.44 (m, 3H), 7.25 (m, 1H), 7.19 (m, 1H), 7.03 (s, 1H), 5.30 (s, 2H), 3.63 (s, 3H), 3.53 (s, 2H).

### EXAMPLE 10B

1-benzyl-5-methoxy-1,2,3,6-tetrahydropyridine-4-carbonitrile

A solution of Example 1B (1.07 g, 5 mmol) in methanol (20 mL) was treated dropwise with trimethylsilyldiazomethane (3.8 mL, 7.5 mmol), stirred for 30 minutes, treated with additional trimethylsilyldiazomethane (3.8 mL), stirred for 1 hour, treated with additional trimethylsilyldiazomethane (3.8 mL), stirred for 1 hour, quenched with glacial acetic acid (3 mL), and concentrated. The concentrate was dissolved in ethyl acetate and treated with saturated sodium bicarbonate. The layers were separated and the aqueous layer was extracted three times with ethyl acetate. The combined organic layers were washed with saturated sodium bicarbonate and brine, dried (Na₂SO₄), filtered, and concentrated. The concentrate was purified by flash column chromatography on silica gel with 20% ethyl acetate/hexanes to provide the desired product (521.5 mg, 46%). MS (DCI/NH₃) m/e 229 (M+H)⁺; ¹H NMR (CDCl₃) δ7.25-7.4 (m, 5H), 3.9 (s, 3H), 3.6 (s, 2H), 3.07 (t, J=2.2 Hz, 2H), 2.56 (t, J=5.5 Hz, 2H), 2.38 (m, 2H).

### EXAMPLE 10C

5-methoxy-1,2,3,6-tetrahydropyridine-4-carbonitrile hydrochloride

A solution of Example 10B (521.5 mg, 2.28 mmol) in 1,2-dichlorethane (10 mL) was treated with 1-chloroethylchloroformate (0.5 mL, 4.57 mmol), heated to reflux for 1.5 hours, cooled to room temperature, and concentrated. The concentrate was treated with methanol (10 mL), heated to 50° C., stirred for 1 hour, cooled to room temperature, concentrated and dried under high vacuum for 18 hours. The concentrate was dissolved in dichloromethane and precipitated with diethyl ether. The solid was collected by filtration, rinsed with hexanes, and dried under high vacuum to provide the desired product (264.5 mg, 66%). MS (DCI/NH₃) m/e 156 (M+H+NH₃); ¹H NMR (CD₃OD) δ4.1 (s, 3H), 3.95 (t, J=1.7 Hz, 2H), 3.33 (t, J=6.1 Hz, 2H), 2.6 (m, 2H).

### EXAMPLE 10D

1-({1-[4-cyano-3-(1-naphthyl)benzyl]-1H-imidazol-5-yl}acetyl)-5-methoxy-1,2,3,6-tetrahydropyridine-4-carbonitrile

A solution of Example 10A (170 mg, 0.45 mmol) in THF (3 mL) at room temperature was treated with 1M LiOH in water (0.5 mL, 0.5 mmol), heated to 50° C. overnight, cooled to room temperature, and concentrated. The concentrate was treated with a mixture of Example 10C (79 mg, 0.45 mmol), HATU (205 mg, 0.54 mmol), DMF (3 mL), and diisopropylethylamine (0.2 mL), stirred overnight, and partitioned between ethyl acetate and saturated sodium bicarbonate. The organic layer was dried (MgSO₄), filtered, and concentrated. The concentrate was purified by HPLC with 1 to 70% acetonitrile/water containing 0.1% TFA to provide the desired product (150 mg, 55%) as the trifluoroacetate salt. MS (ESI(+)) m/e 488 (M+H)⁺; ¹H NMR (400 MHz, CH₃OD) δ9.02 (s, 1H), 7.97 (m, 3H), 7.61−7.44 (m, 8H), 5.58 (m, 2H), 4.22 and 4.14 (two s, 2H), 4.03 (s, 2H) 3.98 and 3.90 (two s, 3H), 3.56 (m, 2H), 2.37−2.22 (m, 2H); Anal. Calcd. for C₃₀H₂₅N₅O₂.1.6TFA.0.53H₂O: C, 58.68; H, 4.10; N, 10.31. Found: C, 58.65; H, 4.04; N, 10.44.

### EXAMPLE 11

1-[3-1-(4-cyanobenzyl)-1 H-imidazol-5-yl]propanoyl]-5-(1-naphthyl)-1,2,3,6-tetrahydropyridine-4-carbonitrile

A mixture of 3-(1-trityl-1H-imidazol-5-yl)propanoic acid (prepared according to the procedure described in Bioorg. Med. Chem. 1998, 6, 2317-2336; 388 mg, 1.01 mmol), Example 1D (274 mg, 1.01 mmol), and EDC (387 mg, 2.02 mmol) in DMF (5 mL) and diisopropylethylamine (0.4 mL) at room temperature was stirred overnight and partitioned between ethyl acetate and saturated sodium bicarbonate. The organic layer was dried (MgSO₄), filtered, and concentrated. The concentrate was treated with a solution of 4-cyanobenzyl bromide (216 mg, 1.1 mmol) in ethyl acetate (10 mL), heated to reflux for 2 days, cooled to room temperature, diluted with diethyl ether (50 mL), and filtered. The filter cake was treated with methanol (10 mL), heated to 50° C. for 2 days, cooled to room temperature, and partitioned between ethyl acetate and saturated sodium bicarbonate. The organic layer was dried (MgSO₄), filtered, and concentrated. The concentrate was purified by HPLC with 1 to 70% acetonitrile/water containing 0.1% TFA to provide the desired product(120 mg, 20%) as the trifluoroacetate salt. ¹H NMR (400 MHz, CH₃OD) δ8.96 (m, 1H), 7.95 (m, 2H), 7.84−7.72 (m, 3H), 7.60−7.39 (m, 7H), 5.62 (two s, 2H), 4.82−4.27 (m, 2H), 3.91−3.76 (m, 2H), 2.89 (s, 2H), 2.85 (m, 1H), 2.79−2.54 (m, 3H). MS (ESI(+)) m/e 472 (M+H)⁺; Anal. Calcd. for C₃₀H₂₅N₅O.1.67TFA.0.73H₂O: C, 59.31; H, 4.20; N, 10.37. Found: C, 59.31; H, 4.18; N, 10.43.

### EXAMPLE 12

1-[2-(4-cyanophenyl)-2-fluoro-2-(1-methyl-1H-imidazol-5-yl)ethyl]-5-(1-naphthyl)-1,2,3,6-tetrahydropyridine-4-carbonitrile

A solution of Example 9B (97 mg, 0.21 mmol) in dichloromethane (2 mL) at −78° C. was slowly treated with DAST (0.22 mL, 1.69 mmol), warmed to room temperature overnight, and partitioned between ethyl acetate and saturated sodium bicarbonate. The organic layer was washed with brine, dried (MgSO₄), filtered, and concentrated. The concentrate was purified by flash column chromatography on silica gel with 0.5% methanol/ethyl acetate with 0.5% NH₄OH to provide the desired product (63 mg, 65%). MS (ESI(+)) m/e 462 (M+H)⁺; ¹H NMR (500 MHz, CDCl₃) δ7.85 (m, 2H), 7.71 (d, J=7.8, 2H), 7.58−7.40 (m, 7H), 7.31 (d, J=3.4, 1H), 7.21 (m, 1H), 3.69−3.45 (m, 2H), 3.38−3.09 (m, 3H), 3.24 (s, 3H), 2.89−2.70 (m, 2H), 2.46 (m, 1H).

### EXAMPLE 13

1(2R)-2-[(4-cyanobenzyl)(methyl)amino]-3-(1-methyl-1H-imidazol-5-yl)propanoyl]-5-(1-naphthyl)-1,2,3,6-tetrahydropyridine-4-carbonitrile

### EXAMPLE 13A

methyl (2R)-2-amino-3-(1-methyl-1H-imidazol-5-yl)propanoate hydrochloride

A suspension of 3-methyl-D-histidine (495 mg, 2.93 mmol) in methanol (15 mL) was treated dropwise with thionyl chloride (0.64 mL), heated to reflux for two hours, cooled to room temperature, and concentrated to provide the desired product. MS (APCI) m/e 184 (M+H)⁺ (free base); ¹H NMR (300 MHz, CH₃OD) δ8.95 (d, J=1.0 Hz, 1H), 7.57 (m, 1H), 4.52 (t, J=7.1 Hz, 1H), 3.94 (s, 3H), 3.88 (s, 3H), 3.56−3.34 (m, 2H).

### EXAMPLE 13B

methyl (2R)-2-[(4-cyanobenzyl)amino]-3-(1-methyl-1H-imidazol-5-yl)propanoate

Example 13A (1.62 g, 6.3 mmol) was treated with a 0° C. mixture of 4-cyanobenzaldehyde (826 mg, 6.3 mmol) and sodium acetate (1.09 g, 13.2 mmol) in methanol (30 mL), stirred for 15 minutes, treated with sodium cyanoborohydride (817 mg, 13 mmol), warmed to room temperature overnight, and partitioned between ethyl acetate and saturated sodium bicarbonate. The organic layer was dried (MgSO₄), filtered, and concentrated. The concentrate was purified by flash column chromatography on silica gel with 10% methanol/ethyl acetate with 0.5% NH₄OH to provide the desired product (1.44 g, 77%). MS (ESI(+)) m/e 299 (M+H)⁺; ¹H NMR (300 MHz, CDCl₃) δ7.59 (d, J=8.4 Hz, 2H), 7.39 (s, 1H), 7.36 (d, J=8.2 Hz, 2H), 6.84 (s, 1H), 3.91 (m, 1H), 3.72 (s, 3H), 3.69 (m, 1H), 3.55 (s, 3H), 3.43 (t, J=7.1 Hz, 1H), 3.02−2.86 (m, 2H), 1.94 (s, 1H).

### EXAMPLE 13C

methyl (2R)-2-[(4-cyanobenzyl)(methyl)amino]-3-(1-methyl-1H-imidazol-5-yl)propanoate

Example 13B (249 mg, 0.84 mmol) was treated with paraformaldehyde (125 mg, 4.18 mmol) in methanol (5 mL), treated with acetic acid (1 drop), stirred for 15 minutes, cooled to 0° C., treated with sodium cyanoborohydride (263 mg, 4.18 mmol), warmed to room temperature overnight, and partitioned between ethyl acetate and saturated sodium bicarbonate. The organic layer was dried (MgSO₄), filtered, and concentrated. The concentrate was purified by flash column chromatography on silica gel with 7% methanol/ethyl acetate with 0.5% NH₄OH to provide the desired product (212 mg, 81%). MS (ESI(+)) m/e 313 (M+H)⁺; ¹H NMR (300 MHz, CDCl₃) δ7.58 (d, J=8.5 Hz, 2H), 7.39 (s, 1H), 7.32 (d, J=8.2 Hz, 2H), 6.82 (s, 1H), 3.85 (m, 1H), 3.74 (s, 3H), 3.71 (m, 1H), 3.54 (t, J=7.5 Hz, 1H), 3.49 (s, 3H), 3.11−3.03 (m, 1H), 2.92−2.84 (m, 1H), 2.32 (s, 3H).

### EXAMPLE 13D

1-[(2R)-2-[(4-cyanobenzyl)(methyl)amino]-3-(1-methyl-1H-imidazol-5-yl)propanoyl]-5-(1-naphthyl)-1,2,3,6-tetrahydropyridine-4-carbonitrile

A solution of Example 13C (198 mg, 0.635 mmol) in THF (5 mL) was treated with 1M LiOH in H₂O (0.7 mL, 0.7 mmol), heated to 50° C. for four hours, cooled to room temperature, and concentrated. The concentrate was treated with a mixture of Example 1D (171 mg, 0.635 mmol) and HATU (300 mg, 0.76 mmol) in DMF (4 mL) and diisopropylethylamine (0.22 mL), stirred overnight, and partitioned between ethyl acetate and saturated sodium bicarbonate. The organic layer was dried (MgSO₄), filtered, and concentrated. The concentrate was purified by flash column chromatography on silica gel with 5% methanol/ethyl acetate with 0.5% NH₄OH, dissolved in acetonitrile (5 mL), treated with iN HCl (20 mL), and lyopholized to provide the desired product (296 mg, 79%) as the dihydrochloride salt. MS (ESI) m/e 515 (M+H)⁺; Anal. Calcd. for C₃₂H₃₀N₆O.2HCl.2H₂O: C, 61.64; H, 5.82; N, 13.48. Found: C, 61.54; H, 5.54; N, 13.77.

### EXAMPLE 14

4-(3-chlorophenyl)-1-{[1-(4-cyanobenzyl)-1H-imidazol-5-yl]methyl}-1,2,5,6-tetrahydropyridine-3-carbonitrile

### EXAMPLE 14A

1-tert-butyl 3-methyl 4-hydroxy-5,6-dihydropyridine-1,3(2H)-dicarboxylate

A biphasic solution of methyl 4-oxo-3-piperidinecarboxylate hydrochloride (3.94 g, 20.4 mmol) in THF (50 mL) and 1N NaOH (50 mL) at 0° C. was treated with di-tert-butyl dicarbonate (5.8 g, 26.5 mmol), warmed to room temperature overnight, and partitioned between ethyl acetate and 5M NH₄Cl. The organic layer was washed with brine, dried (MgSO₄), filtered, and concentrated. The concentrate was purified by flash column chromatography on silica gel with 7% ethyl acetate/hexanes to provide the desired product (3.5 g, 67%). MS (ESI(+)) m/e 258 (M+H)⁺; ¹H NMR (300 MHz, CDCl₃) δ11.97 (s, 1H), 4.05 (s, 2H), 3.78 (s, 3H), 3.57 (t, J=5.8Hz, 2H), 2.37 (t, J=6.1 Hz, 2H), 1.48 (s, 9H).

### EXAMPLE 14B

1-tert-butyl-3-methyl 4-{[(trifluoromethyl)sulfonyl]oxy}-5,6-dihydropyridine-1,3(2H)-dicarboxylate

A solution of Example 14A (958 mg, 3.73 mmol) in dichloromethane (10 mL) at 0° C. was treated with diisopropylethylamine (2 mL) and 2-[N,N-bis(trifluoromethylsulfonyl)amino]pyridine (2.67 g, 7.46 mmol), warmed to room tempeature overnight, treated with additional diisopropylethylamine (2 mL) and 2-[N,N-bis(trifluoromethylsulfonyl)amino]pyridine (2.67 g, 7.46 mmol), stirred for 5 days, and partitioned between ethyl acetate and saturated sodium bicarbonate. The organic layer was washed with brine, dried (MgSO₄), filtered, and concentrated. The concentrate was purified by flash column chromatography on silica gel with 5 to 10% ethyl acetate/hexanes to provide the desired product (1.02 g, 70%). MS (DCI/NH₃) m/e 407 (M+NH₄)⁺; ¹H NMR (300 MHz, CDCl₃) δ4.27 (m, 2H), 3.84 (s, 3H), 3.63 (t, J=5.8 Hz, 2H), 2.52 (m, 2H), 1.48 (s, 9H).

### EXAMPLE 14C

1-tert-butyl 3-methyl 4-(3-chlorophenyl)-5,6-dihydropyridine-1,3(2H)-dicarboxylate

Example 14B (1 g, 2.57 mmol) was treated with a mixture of 3-chlorophenylboronic acid (0.52 g, 3.34 mmol), Pd(PPh₃)₄ (149 mg, 0.13 mmol), and potassium phosphate (1.09 g, 5.14 mmol) in dioxane (20 mL). The mixture was purged with nitrogen, stirred at 85° C. overnight, cooled to room temperature, and partitioned between ethyl acetate and water. The organic layer was washed with brine, dried (MgSO₄), filtered, and concentrated. The concentrate was purified by flash column chromatography on silica gel with 10% ethyl acetate/hexanes to provide the desired product (822 mg, 91%). MS (DCI/NH₃) m/e 369 (M+NH₄)⁺; ¹H NMR (300 MHz, CDCl₃) δ7.28 (m, 2H), 7.13 (m, 1H), 7.01 (m, 1H), 4.25 (m, 2H), 3.60 (t, J=5.4 Hz, 2H), 3.52 (s, 3H), 2.47 (m, 2H), 1.50 (s, 9H).

### EXAMPLE 14D

1-(tert-butoxycarbonyl)-4-(3-chlorophenyl)-1,2,5,6-tetrahydropyridine-3-carboxylic acid

A solution of Example 14C (808 mg, 2.3 mmol) in THF (10 mL) was treated with 1M LiOH (2.8 mL), heated to 50° C. overnight, treated with additional 1M LiOH (1 mL), stirred at 50° C. for 24 hours, cooled to room temperature, treated with 1N HCl (4 mL), and concentrated. The concentrate was partitioned between ethyl acetate and 5M NH₄Cl and the organic layer was dried (MgSO₄), filtered, and concentrated to provide the desired product (754 mg, 97%). MS (DCI/NH₃) m/e 355 (M+NH₄)⁺; ¹H NMR (300 MHz, DMSO) δ7.35 (m, 2H), 7.25 (m, 1H), 7.15 (m, 1H), 4.12 (m, 2H), 3.51 (t, J=5.8 Hz, 2H), 2.43 (m, 2H), 1.44 (s, 9H).

### EXAMPLE 14E

tert-butyl 4-(3-chlorophenyl)-5-cyano-3,6-dihydropyridine-1(2H)-carboxylate

A solution of Example 14D (748 mg, 2.22 mmol) in THF (10 mL) and triethylamine (0.62 mL) at −5° C. was treated with isobutylchloroformate (0.32 mL), stirred for 40 minutes, treated with ammonium hydroxide (1 mL), warmed to room temperature overnight, and partitioned between ethyl acetate and saturated sodium bicarbonate. The organic layer was washed with brine, dried (MgSO₄), filtered, and concentrated. The concentrate was dissolved in dichloromethane (15 mL) and triethylamine (3 mL), cooled to 0° C., treated dropwise with 2.2M phosgene in toluene (6 mL, 13 mmol), warmed to room temperature overnight, and partitioned between ethyl acetate and saturated sodium bicarbonate. The organic layer was dried (MgSO₄), filtered, and concentrated to provide the desired product. MS (DCI/NH₃) m/e 319 (M+H)⁺.

### EXAMPLE 14F

4-(3-chlorophenyl)-1,2,5,6-tetrahydropyridine-3-carbonitrile hydrochloride

Example 14E was treated with 4N HCl (5 mL), stirred for 3.5 hours, and concentrated. The concentrate was dissolved in a minimum of dichloromethane and precipitated with diethyl ether. The supernatant was decanted and the solid was dried to provide the desired product (448 mg, 79% for 3 steps). MS (DCI/NH₃) m/e 219 (M+H)⁺ (free base).

### EXAMPLE 14G

4-(3-chlorophenyl)-1-{[1-(4-cyanobenzyl)-1H-imidazol-5-yl]methyl}-1,2,5,6-tetrahydropyridine-3-carbonitrile

The desired product was prepared by substituting Example 14F (80 mg, 0.31 mmol) and 4-[(5-formyl-1H-imidazol-1-yl)methyl]benzonitrile (66 mg, 0.31 mmol) for Example 2A and 1-methyl-1H-imidazole-5-carbaldehyde, respectively, in Example 2B. Purification by HPLC with 1 to 70% acetonitrile/water containing 0.1% TFA provided the desired product (58 mg, 29%) as the bis-trifluoroacetate salt. MS (ESI(+)) m/e 414 (M+H)⁺; ¹H NMR (500 MHz, CH₃OD) δ9.13 (d, J=0.9 Hz, 1H), 7.76 (d, J=8.4 Hz, 2H), 7.67 (s, 1H), 7.4 (m, 5H), 7.31 (m, 1H), 5.68 (s, 2H), 3.72 (s, 2H), 3.17 (t, J=2.5 Hz, 2H), 2.70 (t, J=5.6 Hz, 2H), 2.43 (m, 2H).

### EXAMPLE 15

4-(3-chlorophenyl)-1-{[1-(4-cyanobenzyl)-1H-imidazol-5-yl]acetyl}-1,2,5,6-tetrahydropyridine-3-carbonitrile

The desired product was prepared by substituting [1-(4-cyanobenzyl)-1H-imidazol-5-yl]acetic acid (prepared according to the procedure described in J. Med. Chem. 1999, 42, 3356-3368; 0.34 mmol) and Example 14F (87 mg, 0.34 mmol) for 3-(1-trityl-1H-imidazol-5-yl)propanoic acid and Example 1D, respectively, in Example 11. Purification by HPLC with 1 to 70% acetonitrile/water containing 0.1% TFA provided the desired product (70 mg, 37%) as the trifluoroacetate salt. MS (ESI(+)) m/e 442 (M+H)⁺; ¹H NMR (500 MHz, CH₃OD) δ8.95 (s, 1H), 7.79 (m, 2H), 7.55−7.41 (m, 7H), 5.54 (m, 2H), 4.36 and 4.28 (two s, 2H), 4.04 (m, 2H), 3.77 (t, J=5.6 Hz, 2H), 2.74 and 2.65 (two m, 2H).

### EXAMPLE 16

methyl 1-{[1-(4-cyanobenzyl)-1H-imidazol-5-yl]methyl}-4-(1-naphthyl)-1,2,5,6-tetrahydropyridine-3-carboxylate

### EXAMPLE 16A

methyl 1-{[1-(4-cyanobenzyl)-1H-imidazol-5-yl]methyl}-4-hydroxy-1,2,5,6-tetrahydropyridine-3-carboxylate

The desired product was prepared by substituting 1-(4-cyanobenzyl)-5-chloromethyl imidazole hydrochloride (prepared according to the procedure described in WO 00/01691; 530 mg, 1.98 mmol) and methyl 4-oxo-3-piperidinecarboxylate hydrochloride (383 mg, 1.98 mmol) for Example 4B and Example 1D, respectively, in Example 4C. Purification by flash column chromatography on silica gel with 2 to 5 to 10% methanollethyl acetate with 0.5% NH₄OH provided the desired product (520 mg, 75%). MS (ESI(+)) m/e 353 (M+H)⁺; ¹H NMR (300 MHz, CDCl₃) δ11.89 (s, 1H), 7.62 (d, J=8.5 Hz, 2H), 7.58 (s, 1H), 7.16 (d, J=8.5 Hz, 2H), 7.02 (s, 1H), 5.33 (s, 2H), 3.73 (s, 3H), 3.39 (s, 2H), 2.98 (s, 2H), 2.53 (t, J=5.7 Hz, 2H), 2.29 (t, J=5.8 Hz, 2H).

### EXAMPLE 16B

methyl 1-{[1-(4-cyanobenzyl)-1H-imidazol-5-yl]methyl}-4-{[(trifluoromethyl)sulfonyl]oxy}-1,2,5,6-tetrahydropyridine-3-carboxylate

Example 16A (493 mg, 1.4 mmol) was treated with a mixture of 2-[N,N-bis(trifluoromethylsulfonyl)amino]pyridine (1.5 g, 4.2 mmol) in dichloromethane (10 mL) and diisopropylethylamine (1.2 mL), stirred for 3 days, and partitioned between ethyl acetate and saturated sodium bicarbonate. The organic layer was washed with brine, dried (MgSO₄), filtered, and concentrated. The concentrate was purified by flash column chromatography on silica el with 1 to 2% methanol/ethyl acetate with 0.2% NH₄OH to provide the desired product (281 mg, 41%). MS (DCI/NH₃) m/e 485 (M+H)⁺; ¹H NMR (300 MHz, CDCl₃) δ7.68 (s, 1H), 7.64 (d, J=8.1 Hz, 2H), 7.16 (d, J=8.1 Hz, 2H), 7.05 (s, 1H), 5.30 (s, 2H), 3.81 (s, 3H), 3.45 (s, 2H), 3.25 (t, J=2.7 Hz, 2H), 2.61 (t, J=5.8 Hz, 2H) 2.37 (m, 2H).

### EXAMPLE 16C

methyl 1-{[1-(4-cyanobenzyl)-1H-imidazol-5-yl]methyl}-4-(1-naphthyl)-1,2,5,6-tetrahydropyridine-3-carboxylate

The desired product was prepared by substituting Eample 16B (280 mg, 0.58 mmol) and 1-naphthylboronic acid (129 mg, 0.75 mmol) for Example 14B and 3-chlorophenylboronic acid, respectively, in Example 14C. Purification by flash column chromatography on silica gel with 1 to 2% methanol/ethyl acetate with 0.2% NH₄OH provided the desired product (137 mg, 51%). MS (ESI) m/e 463 (M+H)⁺; ¹H NMR (300 MHz, CDCl₃) δ7.87−7.75 (m, 2H), 7.67−7.54 (m, 4H), 7.49−7.39 (m, 3H), 7.24 (d, J=8.8 Hz, 2H), 7.10 (m, 2H), 5.44 (s, 2H), 3.55 (s, 2H), 3.44 (m, 2H), 3.22 (s, 3H), 2.72 −2.60 (m, 2H), 2.51−2.37 (m, 2H).

### EXAMPLE 17

4-[(5-{[5-(morpholin-4-ylcarbonyl)-4-(1-naphthyl)-3,6-dihydropyridin-1(2H)-yl]methyl}-1H-imidazol-1-yl)methyl]benzonitrile

### EXAMPLE 17A

1-{[1-(4-cyanobenzyl)-1H-imidazol-5-yl]methyl}-4-(1-naphthyl)-1,2,5,6-tetrahydropyridine-3-carboxylic acid

The desired product was prepared by substituting Example 16C (910 mg, 1.97 mmol) for Example 14C in Example 14D (400 mg, 45%).

### EXAMPLE 17B

4-[(5-{[5-(morpholin-4-ylcarbonyl)-4-(1-naphthyl)-3,6-dihydropyridin-1(2H)-yl]methyl}-1H-imidazol-1-yl)methyl]benzonitrile

The desired product was prepared by substituting Example 17A (102 mg, 0.23 mmol) and morpholine (40 μL, 0.46 mmol) for 3-(1-trityl-1H-imidazol-5-yl)propanoic acid and Example 1D, respectively, in Example 11. Purification by HPLC with 1 to 70% acetonitrile/water containing 0.1% TFA provided the desired product (72 mg, 42%) as the bis-trifluoroacetate salt. MS (ESI(+)) m/e 518 (M+H)⁺; ¹H NMR (500 MHz, CH₃OD) δ9.09 (s, 1H), 7.91−7.75 (m, 6H), 7.50 (m, 5H), 7.28 (dd, J=1.2, 7.2 Hz, 1H), 5.76 (s, 2H), 3.92 (s, 2H), 3.48−2.31 (m, 14H); Anal. Calcd. for C₃₂H₃₁N₅O₂.2.55TFA.0.5H₂O: C, 54.52: H, 4.26; N, 8.57. Found: C, 54.50; H, 4.22; N, 8.66.

### EXAMPLE 18

1-{[(4-cyanophenyl)(1-methyl-1H-imidazol-5-yl)methyl]amino}-5-(1-naphthyl)-1,2,3,6-tetrahydropyridine-4-carbonitrile

### EXAMPLE 18A

tert-butyl 2-(2-methoxy-2-oxoethyl)hydrazinecarboxylate

The desired product was prepared by substituting tert-butyl carbazate and methyl bromoacetate for Example 1D and Example 4B, respectively, in Example 4C, and by stirring the reaction at room temperature for 3 days rather than at 50° C. overnight. MS (DCI/NH₃) m/e 222 (M+H+NH₃)⁺;¹H NMR (CDCl₃) δ6.38 (br s, 1H), 3.75 (s, 3H), 3.66 (s, 2H), 1.45 (s, 9H).

### EXAMPLE 18B

tert-butyl 2-(3-cyanopropyl)-2-(2-methoxy-2-oxoethyl)hydrazinecarboxylate

The desired product was prepared by substituting Example 18A and 4-bromobutyronitrile for Example 1D and Example 4B, respectively, in Example 4C, and by stirring the reaction at 100° C. for 2 days rather than at 50° C. overnight. MS (DCI/NH₃) m/e 289 (M+H+NH₃)⁺; ¹H NMR (CDCl₃) δ6.53 (br s, 1H), 3.75 (s, 3H), 3.70 (s, 2H), 3.01 (br t, 2H), 2.66 (t, 2H), 1.80 (m, 2H), 1.45 (s, 9H).

### EXAMPLE 18C

tert-butyl 4-cyano-5-hydroxy-3,6-dihydropyridin-1(2H)-ylcarbamate

The desired product was prepared by substituting Example 18B for Example 1A in Example 1B. MS (DCI/NH₃) m/e 257 (M+H+NH₃)⁺.

### EXAMPLE 18D

tert-butyl 4-cyano-5-(1-naphthyl)-3,6-dihydropyridin-1(2H)-ylcarbamate

The desired product was prepared by substituting Example 18C for Example 1B in Example 1C. MS (DCI/NH₃) m/e 367 (M+H+NH₃)⁺; ¹H NMR (CDCl₃) δ7.89 (m, 2H), 7.79 (m, 1H), 7.52, (m, 3H), 7.33 (m, 1H), 5.95 (br s, 1H), 3.90 (br m, 2H), 3.31 (dd, 2H), 2.90 (m,1H), 2.70 (m, 1H), 1.45 (s, 9H).

### EXAMPLE 18E

1-amino-5-(1-naphthyl)-1,2,3,6-tetrahydropyridine-4-carbonitrile

Example 18D (335 mg) was treated with 4N HCl in dioxane (5 mL), stirred for 30 minutes, and concentrated to provide the desired product. MS (DCI/NH₃) m/e 267 (M+H+NH₃)⁺.

### EXAMPLE 18F

1-{[(4-cyanophenyl)(1-methyl-1H-imidazol-5-yl)methyl]amino}-5-(1-naphthyl)-1,2,3,6-tetrahydropyridine-4-carbonitrile

The desired product was prepared by substituting Example 18E for Example 1D in Example 4C. The product was purified by flash column chromatography on silica gel with 95/5/1, then 90/10/1 ethyl acetate/ethanol/concentrated NH₄OH, treated with 4M HCl in dioxane (10 mL), stirred for about 4 hours, and lyophilized to provide the desired product as the bis-hydrochloride salt. MS (APCI) m/e 445 (M+H)⁺; ¹H NMR (DMSO-d₆) δ9.04 (d, 1H), 8.00 (m, 2H), 7.87 (m, 2H), 7.70−7.50 (m, 7H), 7.40 (m, 1H), 5.61 (s, 1H), 3.75 (m, along with water peak has 5H, the N—CH₃ and the CH₂ between the ring N and the double bond), 3.20 (br m, 1H), 3.10 (br m, 1H), 2.70 (br m, 1H), 2.55 (br m, 1H); Anal. Calcd. for C₂₈H₂₆Cl₂N₆.0.90H₂O: C, 63.02; H, 5.25; N, 15.75. Found: C, 63.13; H, 5.64; N, 15.39.

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is

1. A compound of formula (I) 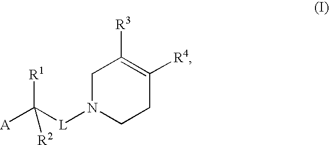
or a therapeutically acceptable salt thereof, wherein
A is selected from the group consisting of aryl and heteroaryl;
L is absent or selected from the group consisting of alkylene, N(R⁵), (CH₂)ₙC(O), (CH₂)ₙC(S), N(R⁵)(CH₂)ₙC(O), CH(NR⁵R⁶)C(O), and (CH₂)ₙSO₂; wherein n is 0-4; and wherein each group is drawn with its left end attached to the carbon bearing R¹ and R² and its right end attached to the nitrogen;
R¹ and R² are independently selected from the group consisting of hydrogen, alkenyl, alkyl, alkynyl, aryl, arylalkenyl, arylalkyl, arylalkynyl, halo, and hydroxy; wherein the aryl and the aryl part of the arylalkenyl and the arylalkynyl can be optionally substituted with one, two, three, four, or five substituents independently selected from the group consisting of alkoxy, alkoxyalkyl, alkoxycarbonyl, alkyl, alkylcarbonyl, cyano, halo, haloalkoxy, haloalkyl, hydroxy, and nitro;
R³ and R⁴ are independently selected from the group consisting of hydrogen, alkoxy, alkoxycarbonyl, alkyl, aminocarbonyl, aryl, carboxy, cyano, halo, heteroaryl, heterocycle, and (heterocycle)carbonyl; and
R⁵ and R⁶ are independently selected from the group consisting of hydrogen, alkyl, aryl, and arylalkyl:

2. The compound of claim 1 wherein A is heteroaryl.

3. The compound of claim 2 wherein L is absent.

4. The compound of claim 3 wherein R¹ and R² are hydrogen.

5. The compound of claim 4 selected from the group consisting of
1-[(1-methyl-1H-imidazol-5-yl)methyl]-5-(1-naphthyl)-1,2,3,6-tetrahydropyridine-4-carbonitrile;
1-{[1-(4-cyanobenzyl)-1H-imidazol-5-yl]methyl}-5-(1-naphthyl)-1,2,3,6-tetrahydropyridine-4-carbonitrile;
4-(3-chlorophenyl)-1-{[1-(4-cyanobenzyl)-1H-imidazol-5-yl]methyl}-1,2,5,6-tetrahydropyridine-3-carbonitrile;
methyl 1-{[1-(4-cyanobenzyl)-1H-imidazol-5-yl]methyl}-4-(1-naphthyl)-1,2,5,6-tetrahydropyridine-3-carboxylate; and
4-[(5-{[5-(morpholin-4-ylcarbonyl)-4-(1-naphthyl)-3,6-dihydropyridin-1(2H)-yl]methyl}-1H-imidazol-1-yl)methyl]benzonitrile.

6. The compound of claim 3 wherein one of R¹ and R² is other than hydrogen.

7. The compound of claim 6 selected from the group consisting of
1-[(4-cyanophenyl)(1-methyl-1H-imidazol-5-yl)methyl]-5-(1-naphthyl)-1,2,3,6-tetrahydropyridine-4-carbonitrile; and
1-[1-(1-methyl-1H-imidazol-5-yl)-3-phenylprop-2-ynyl]-5-(1-naphthyl)-1,2,3,6-tetrahydropyridine-4-carbonitrile.

8. The compound of claim 2 wherein L is (CH₂)ₙC(O).

9. The compound of claim 8 selected from the group consisting of
1-{[1-(4-cyanobenzyl)-1H-imidazol-5-yl]acetyl}-5-(1-naphthyl)-1,2,3,6-tetrahydropyridine-4-carbonitrile;
1-({1-[4-cyano-3-(1-naphthyl)benzyl]-1H-imidazol-5-yl }acetyl)-5-methoxy-1,2,3,6-tetrahydropyridine-4-carbonitrile;
1-{3-[1-(4-cyanobenzyl)-1H-imidazol-5-yl]propanoyl}-5-(1-naphthyl)-1,2,3,6-tetrahydropyridine-4-carbonitrile; and
4-(3-chlorophenyl)-1-{[1-(4-cyanobenzyl)-1H-imidazol-5-yl]acetyl}-1,2,5,6-tetrahydropyridine-3-carbonitrile.

10. The compound of claim 2 wherein L is selected from the group consisting of N(R⁵)(CH₂)ₙC(O) and CH(NR⁵R⁶)C(O).

11. The compound of claim 10 selected from the group consisting of
4-cyano-N-(4-cyanobenzyl)-N-[(1-methyl-1H-imidazol-5-yl)methyl]-5-(1-naphthyl)-3,6-dihydropyridine-1(2H)-carboxamide;
1-({(4-cyanobenzyl)[(1-methyl-1H-imidazol-5-yl)methyl]amino}acetyl)-5-(1-naphthyl)-1,2,3,6-tetrahydropyridine-4-carbonitrile;
1-[(2R)-2-[(4-cyanobenzyl)amino]-3-(1-methyl-1H-imidazol-5-yl)propanoyl]-5-(1-naphthyl)-1,2,3,6-tetrahydropyridine-4-carbonitrile; and
1-[(2R)-2-[(4-cyanobenzyl)(methyl)amino]-3-(1-methyl-1H-imidazol-5-yl)propanoyl]-5-(1-naphthyl)-1,2,3,6-tetrahydropyridine-4-carbonitrile.

12. The compound of claim 2 wherein L is selected from the group consisting of alkylene and N(R⁵).

13. The compound of claim 12 selected from the group consisting of
1-[2-(4-cyanophenyl)-2-hydroxy-2-(1-methyl-1H-imidazol-5-yl)ethyl]-5-(1-naphthyl)-1,2,3,6-tetrahydropyridine-4-carbonitrile;
1-[2-(4-cyanophenyl)-2-fluoro-2-(1-methyl-1H-imidazol-5-yl)ethyl]-5-(1-naphthyl)-1,2,3,6-tetrahydropyridine-4-carbonitrile; and
1-{[(4-cyanophenyl)(1-methyl-1H-imidazol-5-yl)methyl]amino}-5-(1-naphthyl)-1,2,3,6-tetrahydropyridine-4-carbonitrile.

14. A pharmaceutical composition comprising a compound of claim 1 or a therapeutically acceptable salt thereof, in combination with a therapeutically acceptable carrier.

15. A method for inhibiting farnesyltransferase in a patient in recognized need of such treatment comprising administering to the patient a therapeutically acceptable amount of a compound of claim 1, or a therapeutically acceptable salt thereof.

16. A method for treating cancer in a patient in recognized need of such treatment comprising administering to the patient a therapeutically acceptable amount of a compound of claim 1, or a therapeutically acceptable salt thereof.
